(19) United States
(12) Patent Application Publication
(10) Pub. No.: US 2026/0335206 A1
(43) Pub. Date: Oct. 29, 2026

(54) BLEACHING COMPOSITION FOR KERATIN FIBERS

(71) Applicant: KAO CORPORATION, Tokyo (JP)

(72) Inventors: Steven BREAKSPEAR, Darmstadt (DE); Niu JIAN, Darmstadt (DE); Bernd NÖCKER, Darmstadt (DE); Peter BAUER, Darmstadt (DE)

(73) Assignee: KAO CORPORATION, Tokyo (JP)

(21) Appl. No.: 19/484,217

(22) PCT Filed: May 29, 2024
(86) PCT No.: PCT/EP2024/064769

§ 371 Date: Nov. 13, 2025

(30) Foreign Application Priority Data
May 31, 2023 (EP) 23176364.0

(51) Int. Cl. A61K 8/41; A61K 8/22; A61K 8/23; A61K 8/24; A61K 8/25; A61K 8/34; A61K 8/44; A61K 8/46; A61K 8/73; A61K 8/90; A61Q 5/08

(52) U.S. Cl. CPC A61K 8/41; A61K 8/22; A61K 8/23; A61K 8/24; A61K 8/25; A61K 8/342; A61K 8/44; A61K 8/463; A61K 8/73; A61K 8/90; A61Q 5/08; A61K 2800/48; A61K 2800/882

(57) ABSTRACT

A bleaching and/or lightening composition A for keratin fibers, preferably human keratin fibers, more preferably human hair, comprising: a) tris-(hydroxymethyl)-aminomethane and/or 2-amino-2-methyl-1,3-propanediol, and/or their salt(s), and/or their mixtures, b) one or more inorganic alkalizing agents, and c) one or more ammonium or alkali metal or alkaline earth metal salts of persulfates or peroxysulfates.

### FIELD OF THE INVENTION

The present invention is directed to a bleaching and/or lightening compositions for keratin fibers. Additionally, a method for bleaching and/or lightening and a kit-of-parts is disclosed.

### BACKGROUND OF THE INVENTION

Bleaching of keratin fibers is typically desired when customers with dark hair color intend to lighten their hair color. It is also necessary when the customer prefers a color shade that is lighter in comparison to their natural hair color.

A common disadvantage of all bleaching and lightening processes is that they confer a certain degree of damage to keratin fibers, and especially after repeated application, the keratin fibers lack their cosmetic appearance.

Despite the progress in recent years regarding bleaching compositions with high performance and low damage, there still is a need for further improvement.

The prior art has not yet found the ideal solution.

### SUMMARY OF THE INVENTION

Therefore, the first object of the present invention is a bleaching and/or lightening composition A for keratin fibers, preferably human keratin fibers, more preferably human hair, comprising:
a) tris-(hydroxymethyl)-aminomethane and/or 2-amino-2-methyl-1,3-propanediol, and/or their salt(s), and/or their mixtures,
b) one or more inorganic alkalizing agents,
c) one or more ammonium or alkali metal or alkaline earth metal salts of persulfates or peroxysulfates.

The second object of the present invention is a kit-of-parts for bleaching and/or lightening of keratin fibers, preferably human keratin fibers, more preferably human hair, comprising:
a composition A as defined above, and
a composition B comprising one or more oxidizing agent(s), preferably hydrogen peroxide.

The third object of the present invention is a method for bleaching and/or lightening of keratin fibers, preferably human keratin fibers, more preferably human hair, comprising the steps of:
i) mixing a composition A as defined above with a composition B as defined above to yield a ready-to-use composition having a pH in the range of 7 to 12,
ii) applying the ready-to-use composition onto keratin fibers and leaving it,
iii) rinsing-off the ready-to-use composition from keratin fibers.

The fourth object of the present invention is the use of composition A as defined above for reducing the damage during bleaching and/or lightening on keratin fibers, preferably human keratin fibers, more preferably human hair, with the provision that composition A is mixed with a composition B as defined above directly prior to application onto keratin fibers.

### DETAILED DESCRIPTION OF THE INVENTION

The inventors of the present invention unexpectedly found that the presence of compounds in the composition, kit, method and use according to the independent claims resulted in less damage to the keratin fibers while maintaining the same bleaching performance or even increasing the bleaching performance. In addition, the bleached keratin fibers were found to have an improved cosmetic appearance with, for example, an improved touch and a softer feel.

### Bleaching and Lightening Composition

The present invention is directed to a bleaching and/or lightening composition A for keratin fibers, preferably human keratin fibers, more preferably human hair, comprising:
a) tris-(hydroxymethyl)-aminomethane and/or 2-amino-2-methyl-1,3-propanediol, and/or their salt(s), and/or their mixtures,
b) one or more inorganic alkalizing agents,
c) one or more ammonium or alkali metal or alkaline earth metal salts of persulfates or peroxysulfates.

### Compound(s) According to Group a)

The composition of the present invention comprises tris-(hydroxymethyl)-aminomethane and/or 2-amino-2-methyl-1,3-propanediol, and/or their salt(s), and/or their mixtures as compound(s) according to group a).

The most preferred compound according to group a) is tris-(hydroxymethyl)-aminomethane, and/or its salt(s).

It is preferred from the viewpoint of bleaching performance that the total concentration of compound(s) according to group a) is 0.5% by weight or more, preferably 0.75% by weight or more, more preferably 1% by weight or more, calculated to the total weight of composition A.

It is preferred from the viewpoint of bleaching performance and damage that the total concentration of compound(s) according to group a) is 20% by weight or less, preferably 15% by weight or less, more preferably 12.5% by weight of less, calculated to the total weight of composition A.

For attaining the above-mentioned effects, it is preferred that the total concentration of compound(s) according to group a) is in the range of 0.5% to 20% by weight, preferably in the range of 0.75% to 15% by weight, more preferably in the range of 1% to 12.5% by weight, calculated to the total weight of composition A.

### Compound(s) According to Group b)

Composition A of the present invention comprises one or more inorganic alkalizing agents as compound(s) according to group b).

It is preferred from the viewpoint of bleaching performance that the one or more inorganic alkalizing agent according to group b) is/are an alkali metal or alkaline earth metal salt of one or more metasilicates, one or more silicates, one or more disilicates, one or more hydroxides, one or more phosphates, one or more carbonates, one or more bicarbonates, and/or their mixtures, preferably one or more inorganic alkalizing agent according to group b) is/are an alkali metal or alkaline earth metal salt of one or more metasilicates, one or more carbonates, one or more bicarbonates, and/or their mixtures, more preferably one or more inorganic alkalizing agent according to group b) is/are sodium metasilicate, sodium carbonate, sodium bicarbonate, and/or their mixtures.

The furthermore preferred compounds according to group b) are sodium bicarbonate and sodium metasilicate, and/or their mixtures.

In one aspect of the present invention, the most preferred compound according to group b) is sodium bicarbonate.

In another aspect of the present invention, the most preferred compound according to group b) is sodium metasilicate.

It is preferred from the viewpoint of bleaching performance that the total concentration of compound(s) according to group b) is 0.25% by weight or more, preferably 0.5% by weight or more, more preferably 1% by weight or more, furthermore preferably 3% by weight or more, calculated to the total weight of composition A.

It is preferred from the viewpoint of bleaching performance and damage that the total concentration of compound(s) according to group b) is 30% by weight or less, preferably 25% by weight or less, more preferably 20% by weight of less, furthermore preferably 18% by weight or less, calculated to the total weight of composition A.

For attaining the above-mentioned technical effects, it is preferred that the total concentration of compound(s) according to group b) in composition A is in the range of 0.25% to 30% by weight, preferably in the range of 0.5% to 25% by weight, more preferably in the range of 1% to 20% by weight, furthermore preferably in the range of 3% to 18% by weight, calculated to the total weight of composition A.

It is further preferred from the viewpoint of bleaching performance and damage that the weight ratio of compound(s) according to group a) to compound(s) according to group b) is in the range of 0.1 to 10, preferably in the range of 0.2 to 8, more preferably in the range of 0.25 to 6.

### Compound(s) According to Group c)

Composition A of the present invention comprises one or more ammonium or alkali metal or alkaline earth metal salts of persulfates or peroxysulfates as compound(s) according to group c).

Suitable compounds according to group c) is/are sodium persulfate, potassium persulfate, ammonium persulfate, sodium peroxymonosulfate, potassium peroxymonosulfate, and ammonium peroxymonosulfate, and/or their mixtures.

It is preferred from the viewpoint of bleaching performance that the total concentration of compound(s) according to group c) is 1% by weight or more, preferably 2% by weight or more, more preferably 5% by weight or more, calculated to the total weight of composition A.

It is preferred from the viewpoint of bleaching performance and damage that the total concentration of compound(s) according to group c) is 80% by weight or less, preferably 70% by weight or less, more preferably 60% by weight of less, calculated to the total weight of composition A.

For attaining the above-mentioned effects, it is preferred that the total concentration of compound(s) according to group c) in composition A is in the range of 1% to 80% by weight, preferably in the range of 2% to 70% by weight, more preferably in the range of 5% to 60% by weight, calculated to the total weight of composition A.

### Additional Alkalizing Agents

Composition A of the present invention may comprise one or more additional alkalizing agent different from compound(s) according to groups a) and b), preferably the one or more additional alkalizing agent is ammonia and/or its salt(s), an organic alkyl and/or alkanol amines having one hydroxy group, and/or their salt(s), according to the following general structure:

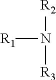

wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and/or their salts, and/or their mixtures.

Suitably, the one or more additional alkalizing agent different from compound(s) according to groups a) and b) is selected from ammonium hydroxide, monoethanolamine, diethanolamine, monoethanol methylamine, monoethanol dimethylamine, diethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine, trimethylamine, triethylamine, 2-amino-2-methylpropanol, and/or its/their salt(s), and/or their mixtures.

It is preferred from the viewpoint of alkalinity and bleaching power that the total concentration of one or more additional alkalizing agent different from compound(s) according to groups a) and b) is 0.25% by weight or more, preferably 0.5% by weight or more, still more preferably 1% by weight or more, calculated to the total weight of composition A.

It is preferred from the viewpoint of alkalinity and stability that the total concentration of one or more additional alkalizing agent different from compound(s) according to groups a) and b) is 30% by weight or less, preferably 25% by weight or less, still more preferably 20% by weight or less, calculated to the total weight of composition A.

For attaining the above-mentioned effects, it is preferred that the total concentration of one or more additional alkalizing agent different from compound(s) according to groups a) and b) is in the range of 0.25% to 30% by weight, preferably 0.5% to 25% by weight, more preferably 1% to 20% by weight, calculated to the total weight of composition A.

### Product Forms

### Aqueous Composition

In one aspect of the present invention, composition A is an aqueous composition comprising water at a total concentration of more than 10% by weight, preferably at 30% by weight or more, more preferably at 50% by weight or more, calculated to the total weight of composition A.

Preferably, from the viewpoint of bleaching efficiency, the pH of composition A is in the range of 7 to 12, more preferably in the range of 8 to 11, still more preferably in the range of 8.5 to 10.5.

The aqueous composition A may be in the form of a solution, a thickened gel, or a oil-in-water emulsion.

### Bleaching Powder

It is preferred from the viewpoint of stability that the total concentration of water in composition A is 10% by weight or less, preferably 5% by weight or less, more preferably 1% by weight or less, still more preferably 0.5% by weight or less, calculated to the total weight of composition A, still more preferably composition A is an anhydrous composition.

The term ‘anhydrous’ does not exclude the presence of bound moisture or crystal water of any of the ingredients. ‘Anhydrous’ means that no water is added to composition A prior to mixing it with composition B as detailed below.

In one aspect of the present invention, composition A is a bleaching powder composition.

For this aspect, it is preferred that composition A comprises one or more pulverulent excipient, preferably one or more organic or inorganic excipient(s).

Suitable pulverulent excipients do not react with any of the compounds of composition A. Suitable ones are, for example, diatomaceous earth, kaolin, bentonite, starch especially corn, tapioca, rice, wheat and potato, nylon powder, montmorillonite, gypsum, sawdust and perlite.

For this aspect of the present invention, it is preferred from the viewpoint of powder stability that the total concentration pulverulent excipients is 10% by weight or more, more preferably 15% by weight or more, still more preferably 20% by weight or more, still more preferably 30% by weight or more, calculated to the total weight of composition A.

It is preferred from the viewpoint of powder stability that the total concentration of pulverulent excipients is 90% by weight or less, more preferably 85% by weight or less, still more preferably 80% by weight or less, calculated to the total weight of composition A.

For attaining the above-mentioned effects, it is preferred that the total concentration of pulverulent excipients is in the range of 10% to 90% by weight, more preferably 15 to 85% by weight, still more preferably in the range of 20% to 80% by weight, still more preferably in the range of 30% to 80% by weight, calculated to the total weight of composition A.

It is further preferred from the viewpoint of cosmetic safety that the bleaching powder is a dust-free bleaching powder. This is commonly achieved by adding one or more oil(s) being liquid at 25 °C and under atmospheric pressure as detailed below for the bleaching paste. For the aspect of the dust-free bleaching powder, the total concentration of one or more oil(s) being liquid at 25 °C and under atmospheric pressure is at 10% by weight or less, calculated to the total weight of composition A.

### Bleaching Paste

In another aspect of the present invention, the composition is a bleach paste composition comprising one or more oil(s) being liquid at 25 °C and under atmospheric pressure.

‘Paste’ within the meaning of the present invention is a dispersion of bleaching compounds in the one or more oil(s) being liquid at 25 °C and under atmospheric pressure as compound(s). In most cases, the dispersion is a suspension of the bleaching compounds.

Suitable one or more oil(s) are one or more hydrocarbon oil(s), one or more ester oil(s) such as fatty acid esters consisting of linear or branched, saturated or unsaturated fatty acids with C₁₂ to C₂₂ being esterified with linear or branched primary alcohols with C₃ to C₁₂, one or more plant oil(s), silicone oil(s), and/or their mixtures.

Suitably, the one or more oil(s) being liquid at 25 °C and atmospheric pressure are mineral oil, paraffinum perliquidum, paraffinum subliquidum, polydecenes, octyl palmitate, isocetyl palmitate, isopropyl palmitate, octyl stearate, oleyl oleate, myristyl myristate, almond oil, avocado oil, coconut oil, corn oil, cottonseed oil, flax seed oil, grapeseed oil, hemp oil, olive oil, palm kernel oil, peanut oil, pumpkin seed oil, rice bran oil, safflower seed oil, sesame seed oil, sunflower seed oil, soybean oil, walnut oil, and/or their mixtures.

Suitable comprises fatty compounds selected from linear or branched, saturated or unsaturated fatty alcohols with C₁₂ to C₂₂ are lauryl alcohol, tridecyl alcohol, myristyl alcohol, pentadecyl alcohol, cetyl alcohol, palmitoleyl alcohol, heptadecyl alcohol, stearyl alcohol, nonadecyl alcohol, arachidyl alcohol, behenyl alcohol, and/or their mixtures, such as cetearyl alcohol.

Suitable examples for fatty acid esters consisting of linear or branched, saturated or unsaturated fatty acids with C₁₂ to C₂₂ being esterified with linear or branched primary alcohols with C₃ to C₁₈ are octyl palmitate, isocetyl palmitate, isopropyl palmitate, octyl stearate, oleyl oleate, and myristyl myristate, as well as their mixtures.

The most preferred oil being liquid at 25 °C and atmospheric pressure is isopropyl myristate.

For this aspect of the present invention, it is preferred from the viewpoint of bleaching paste stability that the total concentration of one or more oil(s) being liquid at 25 °C and atmospheric pressure is more than 1% by weight or more, more preferably 2% by weight or more, still more preferably 5% by weight or more, still more preferably 10% by weight or more, calculated to the total weight of composition A.

It is preferred from the viewpoint of powder stability that the total concentration of one or more oil(s) being liquid at 25 °C and atmospheric pressure is 50% by weight or less, more preferably 40% by weight or less, still more preferably 35% by weight or less, calculated to the total weight of composition A.

For attaining the above-mentioned effects, it is preferred that the total concentration of one or more oil(s) being liquid at 25 °C and atmospheric pressure is in the range of 1% to 55% by weight, preferably in the range of 2% to 50% by weight, more preferably in the range of 5% to 45% by weight, still more preferably in the range of 10% to 45% by weight, calculated to the total weight of composition A.

### Thickening Polymers

It is advantageous from the viewpoint of cosmetic safety that the composition of the present invention further comprises one or more oil-soluble thickening polymer(s), preferably one or more polymer having monomer units comprising styrene, ethylene, propylene, and/or butylene.

Such polymers are offered under the trade name Kraton.

It is preferred from the viewpoint of cosmetic safety that the total concentration of one or more oil-soluble thickening polymer(s) in composition A is 0.5% by weight or more, more preferably 0.75% by weight or more, still more preferably 1% by weight or more, calculated to the total weight of composition A.

It is preferred from the viewpoint of user convenience that the total concentration of one or more oil-soluble thickening polymer(s) in composition A is 10% by weight or less, more preferably 8% by weight or less, still more preferably 6% by weight or less, calculated to the total weight of composition A.

For attaining the above-mentioned effects, it is preferred that the total concentration of one or more oil-soluble thickening polymer(s) is in the range of 0.5% to 10% by weight, preferably in the range of 0.75% to 8% by weight, more preferably in the range of 1% to 6% by weight, calculated to the total weight of composition A.

It is further preferred that composition A comprises one or more water-soluble thickening polymer(s), preferably one or more water-soluble anionic or non-ionic thickening polymer(s), still more preferably one or more polymer comprising units of acrylate, methacrylate, methacrylamide, and/or one or more xanthan gum, one or more dehydroxanthan gum, and/or one or more cellulose polymer such as methylcellulose, ethylcellulose, hydroxyethylcellulose, hydroxypropylmethylcellulose, one or more alginate polymer(s), and/or their salt(s), and/or their mixtures.

Preferably, the thickening polymers are selected from polymers resulting in an aqueous solution and/or aqueous dispersion at pH between 8 and 10 having a viscosity of at least 5,000 mPa·s measured at a polymer concentration of 1% by weight in water at 25 °C, calculated to the total weight of the composition, determined by a Brookfield viscometer, such as at 10 rpm for 1 min, with an appropriate spindle at 25 °C.

Suitable non-ionic thickening polymers are cellulose-based polymers. Suitable examples of cellulose-based polymers are methylcellulose, hydroxymethylcellulose, hydroxyethylcellulose, hydroxypropylcellulose, hydroxyethyl-methylcellulose, and alkylated hydroxyl celluloses such as (C2-C8)-alkylcelluloses or cetyl hydroxyethylcellulose.

Suitable anionic thickening polymers are selected from naturally-based anionic polymers and/or synthetic anionic polymers.

Suitably, the natural anionic polymer(s) may be selected from xanthan gum, dehydroxanthan gum, hydroxypropylxanthan gum, carboxymethyl cellulose and starch based polymers such as vegetable starch and/or their synthetically modified derivatives such as hydroxypropyl starch phosphate. Equally suitable are alginic acids, sodium alginates, ammonium alginates, calcium alginates, gum arabic, and guar gum.

The preferred thickening polymer for the composition of the present invention are natural anionic polymers, more preferably xanthan gum and/or dehydroxanthan gum, from the viewpoint of their biodegradability and low environmental impact.

Preferably, the total concentration of water-soluble thickening polymers of the present invention are 0.1% by weight or more, more preferably 0.25% by weight or more, more preferably 0.5% by weight or more, calculated to the total weight of the composition, from the viewpoint of providing sufficient viscosity to the composition.

Preferably, the total concentration of water-soluble thickening polymers of the present invention are 15% by weight or less, more preferably 12% by weight or more, still more preferably 10% by weight or less, calculated to the total weight of the composition, from the viewpoint of providing sufficient viscosity to the composition and cost of goods.

For attaining the above-mentioned effects, it is preferred that the total concentration of water-soluble thickening polymers in the composition of the present invention is in the range of 0.1% to 15% by weight, preferably 0.25% to 12% by weight, more preferably in the range of 0.5% to 10% by weight, calculated to the total weight of the composition.

### Optional Ingredients

### Surfactants

It is further preferred from the viewpoint of mixability of composition A and wetting of keratin fibers that composition A further comprises one or more surfactant(s), more preferably selected from non-ionic, cationic, anionic, zwitterionic/amphoteric surfactant(s).

Anionic surfactants suitable are in principle known from the cleansing compositions. These are anionic surfactants of the sulfate, sulfonate, carboxylate and alkyl phosphate type, for example, the known C₁₀-C₁₈-alkyl sulfates, and in particular the respective ether sulfates, for example, C₁₂-C₁₄-alkyl ether sulfate, lauryl ether sulfate, especially with 1 to 4 ethylene oxide groups in the molecule, monoglyceride (ether) sulfates, fatty acid amide sulfates obtained by ethoxylation and subsequent sulfatation of fatty acid alkanolamides, and the alkali salts thereof, as well as the salts of long-chain mono- and dialkyl phosphates. Preferred anionic surfactants are alkyl sulphate surfactants especially lauryl sulphate and its salts.

Further suitable surfactants are nonionic surfactants. Non-limiting examples are long-chain fatty acid mono- and dialkanolamides, such as coco fatty acid mono- or diethanolamide and myristic fatty acid mono or diethanolamide, stearic acid mono or diethanolamide, alkyl polyglucosides with an alkyl group of 8 to 18 carbon atoms, and with 1 to 5 glucoside units, sorbitan esters, such as polyethylene glycol sorbitan stearic, palmitic, myristic and lauric acid esters, fatty acid polyglycol esters or poly-condensates of ethyleneoxide and propyleneoxide, as they are on the market, for example, under the trade name “PluronicsR”, as well as fatty alcohol ethoxylates, C₁₀-C₂₂-fatty alcohol ethoxylates, known by the generic terms “Laureth”, “Myristeth”, “Oleth”, “Ceteth”, “Deceth”, “Steareth” and “Ceteareth” according to the CTFA nomenclature, including addition of the number of ethylene oxide molecules, e.g., “Laureth-16”: The average degree of ethoxylation thereby ranges between about 2.5 and about 100, preferably about 10 and about 30.

Suitable amphoteric/zwitterionic surfactants are in particular the various known betaines such as alkyl betaines, fatty acid amidoalkyl betaines and sulfobetaines, for example, lauryl hydroxysulfobetaine; long-chain alkyl amino acids, such as cocoaminoacetate, cocoaminopropionate and sodium cocoamphopropionate and -acetate are also suitable.

Typical cationic surfactants are cetyl trimethyl ammonium chloride, stearyl trimonium chloride, dipalmitoyl dimonium chloride, distearyl dimethyl ammonium chloride, stearamidopropyl trimonuim chloride, dioleoylethyl dimethyl ammonium methosulfate, dioleoylethyl hydroxyethylmonium methosulfate.

It is preferred from the viewpoint of composition stability, wetting of keratin fibers, and ingredient dispersability that the total concentration of surfactants is 0.1% by weight or more, preferably 0.2% by weight or more, still more preferably 0.25% by weight or more, calculated to the total weight of the composition A.

It is preferred from the viewpoint of composition stability, wetting of keratin fibers, and ingredient dispersability that the total concentration of surfactants is 35% by weight or less, preferably 30% by weight or less, still more preferably 25% by weight or less, calculated to the total weight of the composition A.

For attaining the above-mentioned effects, it is preferred that the total concentration of surfactants is in the range of 0.1% to 35% by weight, more preferably 0.2% to 30% by weight, still more preferably 0.25% to 25% by weight, calculated to the total weight of the composition A.

### Kit-of-Parts

The present invention is also directed to a kit-of-parts for bleaching and/or lightening of keratin fibers, preferably human keratin fibers, more preferably human hair, comprising:
a composition A as defined above, and
a composition B comprising one or more oxidizing agent(s), preferably hydrogen peroxide.

The preferred oxidizing agent of composition B is hydrogen peroxide, from the viewpoint of bleaching performance.

Composition B preferably comprises hydrogen peroxide at a total concentration in the range of 0.1% to 20% by weight, preferably 0.25% to 15% by weight, and more preferably 0.5% to 12% by weight, calculated to the total weight of composition B.

In this respect, it is preferred that composition B is an aqueous composition, preferably comprising water at 50% by weight or more, more preferably at 60% by weight or more, still more preferably at 70% by weight or more, calculated to the total weight of composition B.

The pH of composition B preferably is in the range of 1.5 to 5, more preferably in the range of 2 to 4.5, adjusted by suitable acids and bases.

Compositions A and B in this aspect of the present invention are intended to be mixed directly prior to application onto keratin fibers. Upon mixture, the cosmetic form of the ready-to-use composition is a cream, i.e., an emulsion, typically in the form of an oil-in-water emulsion.

Method of Bleaching and/or Lightening

The present invention is also directed to a method for bleaching and/or lightening of keratin fibers, preferably human keratin fibers, more preferably human hair, comprising the steps of:
i) mixing a composition A as defined above with a composition B as defined above to yield a ready-to-use composition having a pH in the range of 7 to 12,
ii) applying the ready-to-use composition onto keratin fibers and leaving it,
iii) rinsing-off the ready-to-use composition from keratin fibers.

It is preferred from the viewpoint of bleaching performance that the pH of the ready-to-use composition as defined in step i) is in the range of 8 to 11, more preferably in the range of 8.5 to 10.5.

It is further preferred from the viewpoint of bleaching performance economy that the leave-on time as defined in step ii) is in the range of 2 min to 45 min, more preferably in the range of 5 min to 40 min, still more preferably in the range of 10 min to 30 min.

The present disclosure is also directed to <1> a bleaching and/or lightening composition A for keratin fibers, preferably human keratin fibers, more preferably human hair, comprising:
a) tris-(hydroxymethyl)-aminomethane and/or 2-amino-2-methyl-1,3-propanediol, and/or their salt(s), and/or their mixtures,
b) one or more inorganic alkalizing agents,
c) one or more ammonium or alkali metal or alkaline earth metal salts of persulfates or peroxysulfates.
<2> The composition according to clause <1> characterized in that one or more compound(s) according to group a) is tris-(hydroxymethyl)-aminomethane, and/or its salt(s).
<3> The composition according to any of the clauses <1> to <2> characterized in that the total concentration of compound(s) according to group a) is in the range of 0.5% to 20% by weight, preferably in the range of 0.75% to 15% by weight, more preferably in the range of 1% to 12.5% by weight, calculated to the total weight of composition A.
<4> The composition according to any of the clauses <1> to <2> characterized in that the total concentration of tris-(hydroxymethyl)-aminomethane, and/or its salt(s) in composition A is in the range of 0.5% to 20% by weight, preferably in the range of 0.75% to 15% by weight, more preferably in the range of 1% to 12.5% by weight, calculated to the total weight of composition A.
<5> The composition according to any of the clauses <1> to <4> characterized in that the one or more inorganic alkalizing agent according to group b) is/are an alkali metal or alkaline earth metal salt of one or more metasilicates, one or more silicates, one or more disilicates, one or more hydroxides, one or more phosphates, one or more carbonates, one or more bicarbonates, and/or their mixtures.
<6> The composition according to any of the clauses <1> to <5> characterized in that the one or more inorganic alkalizing agent according to group b) is/are an alkali metal or alkaline earth metal salt of one or more metasilicates, one or more carbonates, one or more bicarbonates, and/or their mixtures.
<7> The composition according to any of the clauses <1> to <5> characterized in that the one or more inorganic alkalizing agent according to group b) is/are sodium metasilicate, sodium carbonate, sodium bicarbonate, and/or their mixtures.
<8> The composition according to any of the clauses <1> to <7> characterized in that the total concentration of compound(s) according to group b) in composition A is in the range of 0.25% to 30% by weight, preferably in the range of 0.5% to 25% by weight, more preferably in the range of 1% to 20% by weight, furthermore preferably in the range of 3% to 18% by weight, calculated to the total weight of composition A.
<9> The composition according to any of the clauses <1> to <8> characterized in that the one or more compound(s) according to group c) is/are sodium persulfate, potassium persulfate, ammonium persulfate, sodium peroxymonosulfate, potassium peroxymonosulfate, ammonium peroxymonosulfate, and/or their mixtures.
<10> The composition according to any of the clauses <1> to <9> characterized in that the total concentration of compound(s) according to group c) in composition A is in the range of 1% to 80% by weight, preferably in the range of 2% to 70% by weight, more preferably in the range of 5% to 60% by weight, calculated to the total weight of composition A.
<11> The composition according to any of the clauses <1> to <10> characterized in that the weight ratio of compound(s) according to group a) to compound(s) according to group b) is in the range of 0.1 to 10, preferably in the range of 0.2 to 8, more preferably in the range of 0.25 to 6.
<12> The composition according to any of clauses <1> to <11> characterized in that the total concentration of water in composition A is 10% by weight or less, preferably 5% by weight or less, more preferably 1% by weight or less, still more preferably 0.5% by weight or less, calculated to the total weight of composition A, still more preferably composition A is an anhydrous composition.
<13> The composition according to any of the clauses <1> to <12> characterized in that composition A is a bleach powder composition, and preferably comprises one or more pulverulent excipient, more preferably one or more organic or inorganic excipient(s), still more preferably one or more diatomaceous earth, kaolin, bentonite, starch especially corn, tapioca, rice, wheat and potato, nylon powder, montmorillonite, gypsum, sawdust, and perlite, and/or their mixtures.
<14> The composition according to any of the clauses <1> to <13> characterized in that composition A comprises one or more oil(s) being liquid at 25 °C and atmospheric pressure.
<15> The composition according to any clause <14> characterized in that the one or more oil(s) being liquid at 25 °C and atmospheric pressure is/are one or more hydrocarbon oil(s), one or more ester oil(s) such as fatty acid esters consisting of linear or branched, saturated or unsaturated fatty acids with C₁₂ to C₂₂ being esterified with linear or branched primary alcohols with C₃ to C₁₂, one or more plant oil(s), one or more silicone oil(s), and/or their mixtures.
<16> The composition according to any of the clauses <14> to <15> characterized in that the one or more oil(s) being liquid at 25 °C and atmospheric pressure is/are mineral oil, paraffinum perliquidum, paraffinum subliquidum, polydecenes, octyl palmitate, isocetyl palmitate, isopropyl palmitate, octyl stearate, oleyl oleate, myristyl myristate, almond oil, avocado oil, coconut oil, corn oil, cottonseed oil, flax seed oil, grapeseed oil, hemp oil, olive oil, palm kernel oil, peanut oil, pumpkin seed oil, rice bran oil, safflower seed oil, sesame seed oil, sunflower seed oil, soybean oil, walnut oil, and/or their mixtures.
<17> The composition according to any of the clauses <14> to <16> characterized in that the total concentration of one or more oil(s) being liquid at 25 °C and atmospheric pressure is in the range of 1% to 55% by weight, preferably in the range of 2% to 50% by weight, more preferably in the range of 5% to 45% by weight, still more preferably in the range of 10% to 45% by weight, calculated to the total weight of composition A.
<18> The composition according to any of the clauses <1> to <12> and <14> to <17> characterized in that composition A is a is a bleach paste composition.
<19> The composition according to any of the clauses <1> to <11> characterized in that composition A is an aqueous composition comprising water at a total concentration of more than 10% by weight, preferably at 30% by weight or more, more preferably at 50% by weight or more, calculated to the total weight of composition A.
<20> The composition according to any of the clauses <1> to <11> and/or <19> characterized in that the pH of composition A is in the range of 7 to 12, more preferably in the range of 8 to 11, still more preferably in the range of 8.5 to 10.5.
<21> The composition according to any of the clauses <1> to <20> characterized in that composition A comprises one or more water-soluble thickening polymer(s), preferably one or more water-soluble anionic or non-ionic thickening polymer(s), still more preferably one or more polymer comprising units of acrylate, methacrylate, methacrylamide, and/or one or more xanthan gum, one or more dehydroxanthan gum, and/or one or more cellulose polymer such as methylcellulose, ethylcellulose, hydroxyethylcellulose, hydroxypropylmethylcellulose, one or more alginate polymer(s), and/or their salt(s), and/or their mixtures.
<22> The composition according to clause <21> characterized in that the total concentration of one or more water-soluble thickening polymer(s) in composition A is in the range of 0.1% to 15% by weight, preferably 0.25% to 12% by weight, more preferably in the range of 0.5% to 10% by weight, calculated to the total weight of composition A.
<23> The composition according to any of the clauses <1> to <22> characterized in that composition A further comprises one or more surfactant(s), preferably selected from non-ionic, cationic, anionic, zwitterionic/amphoteric surfactant(s).
<24> The composition according to clause <23> characterized in that the total concentration of surfactants is in the range of 0.1% to 35% by weight, more preferably 0.2% to 30% by weight, still more preferably 0.25% to 25% by weight, calculated to the total weight of the composition A.
<25> The composition according to any of the clauses <1> to <24> characterized in that composition A comprises one or more additional alkalizing agent different from compound(s) according to groups a) and b), preferably the one or more additional alkalizing agent is ammonia and/or its salt(s), an organic alkyl and/or alkanol amines having one hydroxy group, and/or their salt(s) according to the following general structure:

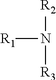

wherein R1, R2, and R3 are independently selected from H, linear C1-C6 alkyl which may be substituted with one hydroxyl group, or branched C3-C12 alkyl or alkanol, wherein at least one of R1, R2, or R3 is different from H, and/or their salts, and/or their mixtures.
<26> The composition according to clause <25> characterized in that the one or more additional alkalizing agent different from compound(s) according to groups a) and b) is/are selected from ammonium hydroxide, monoethanolamine, diethanolamine, monoethanol methylamine, monoethanol dimethylamine, diethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine, trimethylamine, triethylamine, 2-amino-2-methylpropanol, and/or its/their salt(s), and/or their mixtures.
<27> The composition according to any of the clauses <25> to <26> characterized in that the total concentration of one or more additional alkalizing agent different from compound(s) according to groups a) and b) is in the range of 0.25% to 30% by weight, preferably 0.5% to 25% by weight, more preferably 1% to 20% by weight, calculated to the total weight of composition A.
<28> The composition according to any of the clauses <1> to <27> characterized in that composition A comprises one or more oil-soluble thickening polymer(s).
<29> The composition according to clause <28> characterized in that one or more oil-soluble thickening polymer(s) have monomer units comprising styrene, ethylene, propylene, and/or butylene.
<30> The composition according to any of the clauses <28> to <29> characterized in that the total concentration of one or more oil-soluble thickening polymer(s) is in the range of 0.5% to 10% by weight, preferably in the range of 0.75% to 8% by weight, more preferably in the range of 1% to 6% by weight, calculated to the total weight of composition A.

The present disclosure is also directed to <31> a kit-of-parts for bleaching and/or lightening of keratin fibers, preferably human keratin fibers, more preferably human hair, comprising:
a composition A as defined in any of the clauses <1> to <30>, and
a composition B comprising one or more oxidizing agent(s), preferably hydrogen peroxide.
<32> The kit according to clause <31> characterized in that the total concentration of oxidizing agents, preferably the total concentration of hydrogen peroxide, in Composition B is in the range of 0.1% to 20% by weight, preferably 0.25% to 15% by weight, and more preferably 0.5% to 12% by weight, calculated to the total weight of composition B.
<33> The kit according to any of the clauses <31> to <32> characterized in that the pH of composition B is in the range of 1.5 to 5, preferably in the range of 2 to 4.5, adjusted by suitable acids and bases.
<34> The kit according to any of the clauses <31> to <33> characterized in that compositions A and B are kept separate and are mixed directly prior to application onto keratin fibers.

The present disclosure is also directed to <35> a method for bleaching and/or lightening of keratin fibers, preferably human keratin fibers, more preferably human hair, comprising the steps of:
i) mixing a composition A as defined in any of the clauses <1> to <34> with a composition B as defined in any of the clauses <31> to <34> to yield a ready-to-use composition having a pH in the range of 7 to 12,
ii) applying the ready-to-use composition onto keratin fibers and leaving it,
iii) rinsing-off the ready-to-use composition from keratin fibers.
<36> The method according to clause <35> characterized in that the pH of the ready-to-use composition as defined in step i) is in the range of 8 to 11, preferably in the range of 8.5 to 10.5.
<37> The method according to any of the clauses <35> to <36> characterized in that the leave-on time as defined in step ii) is in the range of 2 min to 45 min, preferably in the range of 5 min to 40 min, more preferably in the range of 10 min to 30 min.

The following examples are to illustrate the present invention, but not to limit it.

### EXAMPLES

TABLE 1
The following compositions were prepared by conventional mixing techniques.

| | | Comp. Ex. 1 | Inv. Ex. 1 | Inv. Ex. 2 | Inv. Ex. 3 | Inv. Ex. 4 | Inv. Ex. 5 |
|---|---|---|---|---|---|---|---|
| | Ingredients | [% by weight] | | | | | |
| Comp. | Sodium persulfate | 50.0 | 50.0 | 50.0 | 50.0 | 50.0 | 50.0 |
| A | Sodium metasilicate | 10.0 | — | 2.0 | 4.0 | 6.0 | 8.0 |
| | Tris-(hydroxymethyl)- | — | 10.0 | 8.0 | 6.0 | 4.0 | 2.0 |
| | aminomethane | | | | | | |
| | Tetrasodium EDTA | 3.5 | 3.5 | 3.5 | 3.5 | 3.5 | 3.5 |
| | Diatomaceous earth | Ad 100.0 | | | | | |
| Comp. | Hydrogen peroxide | 9.0 | | | | | |
| B | Phosphoric acid | Ad pH 3.5 | | | | | |
| | Water | Ad 100.0 | | | | | |
| Mix | Mix comp. A:comp. B | 1:1.4 | 1:1.4 | 1:1.4 | 1:1.4 | 1:1.4 | 1:1.4 |
| | NaOH in mixture | Ad pH 10.0 ± 0.2 | | | | | |
| Results | ΔL* | 33.20 | 32.71 | 33.10 | 31.29 | 33.03 | 34.29 |
| | 25% index/MPa_{(untreated)} | 76.85 | 76.85 | — | — | — | — |
| | 25% index/MPa_{(bleached)} | 58.70 | 73.13 | — | — | — | — |
| | 2% index/MPa_{(untreated-bleached)} | 18.15 | 3.72 | — | — | — | — |
| | Damage_{(untreated-bleached)}/ΔL* | 0.55 | 0.11 | — | — | — | — |

TABLE 2
The following compositions were prepared by mixing the powder
components and dispersing them in isopropyl myristate:

| | | | Inventive | Comparative |
|---|---|---|---|---|
| | | | ex. 5 | ex. 2 |
| Ingredients | | | % by weight | |
| Compound | a) | Potassium persulfate | 50.0 | 50.0 |
| | b) | Tris-(hydroxymethyl)- | 3.0 | — |
| | | aminomethane | | |
| | — | Sodium metasilicate | 11.0 | 14.0 |
| | — | Sodium lauryl sulfate | 5.0 | 5.0 |
| | — | Styrene-Ethylen- | 3.5 | 3.5 |
| | | Propylen-block | | |
| | | copolymer* | | |
| | — | Xanthan gum | 0.4 | 0.4 |
| | — | Tetrasodium EDTA | 2.0 | 2.0 |

| | | |
|---|---|---|
| — | Isopropyl myristate | Ad 100.0 |

*Kraton G1701 sold by Kraton Polymers LLC

The bleach oil composition from above was mixed with the following oxidizing composition in a weight ratio of 1:1.4 directly prior to application onto hair:

| | % by weight |
|---|---|
| Hydrogen peroxide | 9.0 |
| Phosphoric acid | ad pH 3.5 |
| Water | ad 100.0 |

The resulting ready-to-use mixture had a pH around 9.5.

The following bleaching and coloring results were obtained:

| | | Inventive | Comparative |
|---|---|---|---|
| Hair type | | ex. 5 | ex. 2 |
| L6 | ΔL* | 43.49 | 34.56 |
| hair | 2% index/MPa_{(untreated)} | 49.40 | 49.40 |
| | 2% index/MPa_{(bleached)} | 26.29 | 24.84 |
| | 2% index/MPa_{(untreated-bleached)} | 23.11 | 24.56 |
| | Damage_{(untreated-bleached)}/ΔL* | 0.53 | 0.71 |
| | Feel | Smooth, easy | Rough, |
| | | combing | difficult |
| | | | to comb |
| | | | through |

### Methods

### Hair Bleaching

Human level 6 hair (21 cm long, 2 g per bundle) were purchased from International Hair Importers, Glendale, NY, USA. The hair was shampooed prior to treatment.

2 g of each of the compositions from above were applied onto a hair streak and left for 30 min at 50 °C. Then the hair streaks were rinsed off with water, shampooed, and blow-dried.

### Lightening Measurements

L values were measured before and after bleaching with a Datacolor 45G instrument. ΔL was calculated by subtracting the L value before bleaching from the L value after bleaching.

### Damage Measurements

Damage was assessed, for twenty-five individual European hair fibres per treatment, via tensile test using an MTT680 instrument (Dia-Stron Limited, Andover, UK), in the wet condition. The initial sample length used was thirty millimetres and the strain rate was 100 percent per minute. The damage value is expressed as the stress (in MPa) at two percent or twenty five percent strain, where the higher number damage/lift (Damage_{(untreated-bleached)}/ΔL*) indicates higher damage.

The following examples are within the scope of the present invention.

Inv. Ex. 6

### Composition A

| | % by weight |
|---|---|
| Sodium persulfate | 20.0 |
| Ammonium persulfate | 15.0 |
| Tris-(hydroxymethyl)-aminomethane | 5.0 |
| Sodium metasilicate | 5.0 |
| Sodium lauryl sulfate | 3.0 |
| Tetrasodium EDTA | 1.5 |
| Diatomaceous earth | ad 100.0 |

The bleach powder composition is mixed with the oxidative composition B of table 1 in a weight ratio of 1:1.4 (composition A:composition B).

Inv. Ex. 7

### Composition A

| | % by weight |
|---|---|
| Sodium persulfate | 20.0 |
| Ammonium persulfate | 15.0 |
| Tris-(hydroxymethyl)-aminomethane | 5.0 |
| Sodium metasilicate | 5.0 |
| Sodium lauryl sulfate | 3.0 |
| Tetrasodium EDTA | 1.5 |
| Styrene-Ethylen-Propylen-block copolymer | 1.0 |
| Xanthan gum | 0.5 |
| Isopropyl myristate | ad 100.0 |

The bleach powder composition is mixed with the oxidative composition B of table 1 in a weight ratio of 1:1.4 (composition A:composition B).

Inv. Ex. 8

### Composition A

| | % by weight |
|---|---|
| Tris-(hydroxymethyl)-aminomethane | 10.0 |
| Sodium bicarbonate | 2.0 |
| Cetearyl alcohol | 6.0 |
| Mineral oil | 2.0 |
| Sodium lauryl sulfate | 3.0 |
| Tetrasodium EDTA | 0.25 |
| Xanthan gum | 0.5 |
| Water | ad 100.0 |

The bleaching composition is mixed with the oxidative composition B of table 1 in a weight ratio of 1:1.4 (composition A:composition B).

1. A bleaching and/or lightening composition A for keratin fibers, comprising:
a) at least one selected from the group consisting of tris-(hydroxymethyl)-aminomethane, 2-amino-2-methyl-1,3-propanediol, a salt thereof, and a mixture thereof;
b) at least one inorganic alkalizing agent; and
c) at least one selected from the group consisting of an ammonium salt of a persulfate or a peroxysulfate, an alkali metal salt of a persulfate or a peroxysulfate, and an alkaline earth metal salt of a persulfate or a peroxysulfate.

2. The composition according to claim 1, wherein the at least one compound according to a) comprises tris-(hydroxymethyl)-aminomethane and/or a salt thereof.

3. The composition according to claim 1, wherein the total concentration of the at least one compound according to a) is in the range of 0.5% to 20% by weight calculated to the total weight of composition A.

4. The composition according to claim 1, wherein the at least one inorganic alkalizing agent according to b) comprises at least one alkali metal or alkaline earth metal salt selected from the group consisting of a metasilicate, a silicate, a disilicate, a hydroxide, a phosphate, a carbonate, a bicarbonate, and a mixture thereof.

5. The composition according to claim 1, wherein the total concentration of the at least one inorganic alkalizing agent according to b) in composition A is in the range of 0.25% to 30% by weight, calculated to the total weight of composition A.

6. The composition according to claim 1, wherein the at least one compound according to c) comprises at least one selected from the group consisting of sodium persulfate, potassium persulfate, ammonium persulfate, sodium peroxymonosulfate, potassium peroxymonosulfate, ammonium peroxymonosulfate, and a mixture thereof.

7. The composition according to claim 1, wherein the weight ratio of the at least one compound according to a) to the at least one compound according to b) is in the range of 0.1 to 10.

8. The composition according to claim 1, wherein the total concentration of water in composition A is 10% by weight or less, calculated to the total weight of composition A.

9. The composition according to claim 1, wherein the composition A is a bleach powder composition, and the composition A further comprises:
at least one selected from the group consisting of diatomaceous earth, kaolin, bentonite, starch, nylon powder, montmorillonite, gypsum, sawdust, perlite, and a mixture thereof.

10. The composition according to claim 1, wherein the composition A further comprises;
at least one oil being liquid at 25 °C and atmospheric pressure selected from the group consisting of mineral oil, paraffinum perliquidum, paraffinum subliquidum, polydecene, octyl palmitate, isocetyl palmitate, isopropyl palmitate, octyl stearate, oleyl oleate, myristyl myristate, almond oil, avocado oil, coconut oil, corn oil, cottonseed oil, flax seed oil, grapeseed oil, hemp oil, olive oil, palm kernel oil, peanut oil, pumpkin seed oil, rice bran oil, safflower seed oil, sesame seed oil, sunflower seed oil, soybean oil, walnut oil, and a mixture thereof.

11. The composition according to claim 1, wherein the composition A is a bleach paste composition.

12. The composition according to claim 1, wherein the composition A is an aqueous composition comprising water at a total concentration of more than 10% by weight calculated to the total weight of composition A.

13. The composition according to claim 1, wherein the composition A further comprises:
at least one water-soluble thickening polymer selected from the group consisting of a water-soluble anionic polymer and a non-ionic thickening polymer.

14. A kit-of-parts for bleaching and/or lightening of keratin fibers, comprising:
the composition A of claim 1; and
a composition B comprising hydrogen peroxide as an oxidizing agent.

15. A method for bleaching and/or lightening of keratin fibers, the method comprising:
i) mixing the composition A of claim 1 with a composition B comprising hydrogen peroxide as an oxidizing agent to yield a ready-to-use composition having a pH in the range of 7 to 12;
ii) applying the ready-to-use composition onto keratin fibers; and
iii) rinsing-off the ready-to-use composition from the keratin fibers.

16. The composition according to claim 1, wherein the total concentration of the compound according to c) in the composition A is in the range of 1% to 80% by weight, calculated to the total weight of composition A.

17. The composition according to claim 1, wherein the composition A is an anhydrous composition.

18. The composition according to claim 1, wherein a pH of composition A is in the range of 7 to 12.

19. The composition according to claim 1, wherein the composition A further comprises:
at least one water-soluble thickening polymer selected from the group consisting of a polymer comprising units of acrylate, methacrylate, and/or methacrylamide, xanthan gum, dehydroxanthan gum, a cellulose polymer, an alginate polymer, a salt thereof, and a mixture thereof.

20. The composition according to claim 1, wherein the composition A further comprises:
at least one oil being liquid at 25 °C and atmospheric pressure at a total concentration in the range of 1 to 55% by weight, calculated to the total weight of composition A.
